(19) United States
(12) Patent Application Publication
(10) Pub. No.: US 2015/0218539 A1
(43) Pub. Date: Aug. 6, 2015

(54) METHODS OF USING THE CALCINEURIN A VARIANT CNAB1 FOR THE TREATMENT OF CARDIAC HYPERTROPHY

(71) Applicants: CNIC FUNDACÍON CENTRO NACIONAL DE INVESTIGACIONES CARDIOVASCULARES CARLOS III, Madrid (ES); EMBL EUROPEAN MOLECULAR BIOLOGY LABORATORY, Heidelberg (DE)

(72) Inventors: Enrique Lara Pezzi, Madrid (ES); Marina López Olaneta, Madrid (ES); Maria Villalba Orero, Madrid (ES); Jesús Gómez Salinero, Madrid (ES); Nadia Rosenthal, Heidelberg (DE)

(73) Assignee: EMBL EUROPEAN MOLECULAR BIOLOGY LABORATORY, Heidelberg (DE)

(21) Appl. No.: 14/422,112

(22) PCT Filed: Aug. 16, 2013
(86) PCT No.: PCT/EP2013/067140

§ 371 Date: Feb. 17, 2015

(30) Foreign Application Priority Data
Aug. 17, 2012 (EP) 12382329.6

(51) Int. Cl. C12N 9/16; C12Q 1/68; A61K 35/34; A61K 38/46; A61K 31/713

(52) U.S. Cl. CPC C12N 9/16; A61K 38/465; A61K 31/713; A61K 35/34; C12Q 1/6876; C12Y 301/03; C12Q 2600/136; C12Q 2600/158

(57) ABSTRACT

The present invention relates to an activator of the calcineurin subunit Aβ1 isofomi (CₙAβ1) or of the C-terminal domain of the calcineurin subunit Aβ1 isofomi (CₙAβ1) for the production of a medicament for the modulation of myocardial growth without adversely affecting contractile function.

### TECHNICAL FIELD OF THE INVENTION

The present invention refers to the biotechnological field, more particularly to the use of the calcineurin subunit Aβ1 isoform (CnAβ1) for the treatment or prevention of pathologic cardiac hypertrophy.

### BACKGROUND OF THE INVENTION

The following discussion of the background of the invention is merely provided to aid the reader in understanding the invention, and is not admitted to describe or constitute prior art to the present invention.

Cardiac hypertrophy is a thickening of the heart muscle (myocardium) which results in a decrease in size of the chamber of the heart, including the left and right ventricles. Although hypertrophy in response to pathologic signaling has traditionally been considered an adaptive response required to sustain cardiac output in the face of stress, prolonged hypertrophy is associated with a significant increase in the risk for sudden death or progression to heart failure, independent of the underlying cause of hypertrophy, suggesting that the hypertrophic process is not entirely beneficial. This notion is further supported by observations in clinical trials, that inhibition or even regression of cardiac hypertrophy by certain drugs, such as angiotensin-converting enzyme (ACE) inhibitors, lowers the risk for several endpoints including death and progression to heart failure, whereas persistence of cardiac hypertrophy (despite similar blood pressure changes) predicts an adverse outcome.

These findings provide us with information that while hypertrophy can eventually normalize wall tension, it is associated with an unfavorable outcome and threatens affected patients with sudden death or progression to overt heart failure. Accumulating evidence from studies in human patients and animal models suggests that in most instances hypertrophy is not a compensatory response to the change in mechanical load, but rather is a maladaptive process. Accordingly, modulation of myocardial growth without adversely affecting contractile function is increasingly recognized as a potentially auspicious approach in the prevention and treatment of heart failure.

We herein propose a novel antihypertrophic strategy for the prevention and treatment of pathologic cardiac hypertrophy.

### BRIEF DESCRIPTION OF THE INVENTION

The present invention relates to an activator of the calcineurin subunit Aβ1 isoform (CnAβ1) or of the C-terminal domain of the calcineurin subunit Aβ1 isoform (CnAβ1) for the production of a medicament for the modulation of myocardial growth without adversely affecting contractile function.

Thus, a first aspect of the invention refers to a composition (from hereinafter composition of the invention) comprising a compound (from hereinafter activator compound of the invention) capable of
a. increasing the intracellular concentration of the calcineurin subunit Aβ1 isoform (CnAβ1) in the cardiomyocytes of a subject relative to that observed in the absence of the compound; and/or
b. increasing the intracellular concentration of a peptide comprising a fragment of the calcineurin subunit Aβ1 isoform (CnAβ1) in the cardiomyocytes of a subject relative to that observed in the absence of the compound, wherein said fragment consist of the C-terminal domain of the calcineurin subunit Aβ1 isoform (CnAβ1); for its use in the treatment of pathologic cardiac hypertrophy.

In a preferred embodiment of the first aspect of the invention, said compound is capable of increasing the intracellular expression of the calcineurin subunit Aβ1 isoform (CnAβ1) in the cardiomyocytes of a subject relative to that observed in the absence of the compound.

In a more preferred embodiment of the first aspect of the invention, the calcineurin subunit Aβ1 isoform (CnAβ1) is the human calcineurin subunit Aβ1 isoform (CnAβ1).

In another preferred embodiment of the first aspect of the invention, the pathologic cardiac hypertrophy is not induced by an ischemic heart disease with acute thrombocytic coronary occlusion such as myocardial infarction. Preferably the pathologic cardiac hypertrophy is induced by pressure overload. More preferably, the pathologic cardiac hypertrophy is induced by pressure overload caused by aortic valve stenosis, chronic hypertension (hypertensive cardiomyopathy), pulmonary hypertension or hypertrophic cardiomyopathy.

In yet another preferred embodiment of the first aspect of the invention, the pathologic cardiac hypertrophy is induced by a disease selected from the list consisting of hypertension, mechanical load, myocardial infarction, cardiac arrhythmias, endocrine disorders, and genetic mutations in cardiac contractile protein genes.

The present invention also provides for an activator peptide compound which comprises:
a) the sequence of SEQ ID No 1;
b) a fragment of (a), which at least comprises SEQ ID No 2; or
c) a variant of (a) or (b) which is at least 70% homologous to SEQ ID No 1 or to a fragment of SEQ ID No 1 which at least comprises SEQ ID No 2, more preferably at least 80%, 85%, 90%, 95%, 97%, 98% or 99% homologous to SEQ ID No 1 or to a fragment of SEQ ID No 1 which at least comprises SEQ ID No 2.
   for its use in the treatment of pathologic cardiac hypertrophy.

In a preferred embodiment of the previous aspect of the invention, the activator compound is a peptide which comprises SEQ ID No 1 or a peptide comprising a fragment of SEQ ID No consisting of SEQ ID No 2.

In another preferred embodiment of the first aspect of the invention, the activator compound is a polynucleotide (from hereinafter polynucleotide of the invention) having a sequence selected from:
a) a DNA sequence encoding SEQ ID No 1 or the complementary sequence thereto;
b) a DNA sequence encoding a peptide comprising a fragment of SEQ ID No 1 consisting of SEQ ID No 2, or the complementary sequence thereto;
c) a sequence which selectively hybridises to a said sequence (a) or (b);
d) a DNA sequence encoding a peptide sequence which is at least 70% homologous to SEQ ID No 1, more preferably at least 80%, 85%, 90%, 95%, 97%, 98% or 99% homologous to SEQ ID No 1; or
e) a DNA sequence encoding a peptide sequence comprising an amino acid sequence which is at least 70% homologous to a fragment of SEQ ID No 1 consisting of SEQ ID No 2, more preferably at least 80%, 85%, 90%, 95%, 97%, 98% or 99% homologous to a fragment of SEQ ID No 1 consisting of SEQ ID No 2.

In a further preferred embodiment of the first aspect of the invention, the above mentioned polynucleotide of the invention (as an activator compound) is a DNA sequence encoding SEQ ID No 1 or the complementary sequence thereto. In another preferred embodiment of the first aspect of the invention, the polynucleotide of the invention is a DNA sequence encoding a peptide comprising a fragment of SEQ ID No 1 consisting of SEQ ID No 2, or the complementary sequence thereto.

In yet another preferred embodiment of the first aspect of the invention, the activator compound is a mRNA polynucleotide (from hereinafter mRNA polynucleotide of the invention) having a sequence selected from:
a) an mRNA sequence encoding SEQ ID No 1;
b) an mRNA sequence encoding a peptide fragment of SEQ ID No 1 consisting of SEQ ID No 2; )
c) an mRNA sequence encoding a peptide sequence which is at least 70% homologous to SEQ ID No 1, more preferably at least 80%, 85%, 90%, 95%, 97%, 98° A or 99%© homologous to SEQ ID No 1; or
d) an mRNA sequence encoding a peptide sequence which is at least 70% homologous to a fragment of SEQ ID No 1 consisting of SEQ ID No 2, more preferably at least 80%, 85%, 90%, 95%, 97%, 98% or 99% homologous to a fragment of SEQ ID No 1 consisting of SEQ ID No 2.

Another preferred aspect of the invention refers to a vector comprising the polynucleotides of the invention. In a further preferred aspect of the invention said vector is a viral vector. In an even further aspect of the invention the viral vector is selected from the list consisting of adenoviral, retroviral or Adeno-Associated viral Vectors.

In another aspect of the invention, the composition of the invention comprises cells that recombinantly express CnAβ1 or a peptide comprising the C-terminal domain of the calcineurin subunit Aβ1 isoform (CnAβ1).

In a further aspect of the invention, the composition of the invention is a pharmaceutical composition (from hereinafter pharmaceutical composition of the invention), optionally comprising a pharmaceutically acceptable carrier.

In a still further aspect of the invention, the pharmaceutical composition of the invention is used in combination therapy with a further active pharmaceutical ingredient.

A further aspect of the invention refers to a method for screening a compound for the ability to activate CnAβ1, comprising:
a. contacting a cell with a compound suspected to activate CnAβ1;
b. assaying the contents of the cells to determine the amount and/or biological activity of CnAβ1; and
c. comparing the determined amount and/or biological activity of CnAβ1 to a predetermined level, wherein a change of said amount and/or biological activity of CnAβ1 is indicative for a compound that activates CnAβ1.

In a preferred aspect of the invention, the cell mentioned in the screening method above is a cardiomyocyte.

A yet further aspect of the invention refers to a method for producing a compound capable of activating CnAβ1, which comprises:
a. Screening for a compound for the ability to activate CnAβ1 according to the previous aspect of the invention; and
b. Providing the compound identified in step (a).

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows an schematic representation of CnA isoforins, A, CnAalpha, CnAbeta2 and CnAgamma (not shown) share a catalytic domain, a CnB-interacting region, a CaM-binding domain and an autoinhibitory domain. CnAbeta1 is a naturally-occurring splicing variant of CnAbeta2, with an alternative C-terminal domain that has no homology with the autoinhibitory domain. CnAalpha* and CnAbeta* represent constitutively activated CnA isoforms in which the autoinhibitory domain has been artificially deleted. B, CnAbeta1 is generated by alternative splicing of the CnAbeta mRNA through insertion of intron 12-13 after exon 12.

FIG. 2 shows that overexpression of CnAbeta1 in cardiomyocytes attenuates heart enlargement following transaortic constriction (TAC).

FIG. 3 shows a TAC induced in wild type (WT) and αMHC-CnAbeta1 mice wherein corrected LV mass (A), systolic left ventricle posterior wall thickness (B, LVPWs) and systolic interventricular septum thickness (C, IVSs) were measured by echocardiography 21 days later.

FIG. 4 shows that overexpression of CnAbeta1 in the hypertrophic heart improves cardiac function.

FIG. 5 shows a TAC induced in wild type (WT) and αMHC-CnAbeta1 mice wherein RNA was isolated from the left ventricle 21 days later. Atrial natriuretic peptide (A, ANF) and alpha skeletal actin (B, Acta1) mRNA were measured by qRT-PCR.

FIG. 6 shows a TAC induced in wild type (WT) and αMHC-CnAbeta1 mice wherein RNA was isolated 21 days later from the left ventricle. Collagen I α1 (A, Col1a1) and lysyl oxidase (B, Lox) mRNA were measured by qRT-PCR.

FIG. 7 shows SEQ ID No 1 which represents the amino acid sequence of the human CnAbeta1 protein and SEQ ID No 2 which represents the amino acid sequence of the C-terminal domain of the human CnAbeta1 protein.

FIG. 8 shows a scheme representing a double transgenic mouse line (rtTA-CnAbeta1) in which CnAbeta1 overexpression is induced in a cardiomyocyte-specific manner by administering doxycycline in the diet. Expression of the reverse Tet transactivator (rtTA) is controlled by the myosin light chain 2v promoter from Xenopus tropicalis to ensure cardiomyocyte-specific expression. Activation of the rtTA with doxycicline allows activation of the Tet operator, which leads to overexpression of CnAbeta1 only in cardiomyocytes. Pressure overload cardiac hypertrophy was induced by transaortic constriction (TAC). A sham operation procedure (top group) was used as a negative control. CnAbeta1 overexpression was induced in rtTA-CnAβ1 mice by administering doxycycline in the diet (Dox) for 21 days, starting 21 days after TAC, Echocardiography was carried out 42 days after TAC.

FIG. 9 shows a TAC induced in rtTA-CnAbeta1 mice wherein doxycycline (Dox) was administered in the diet for 21 days, starting 21 days after TAC, and corrected LV mass was measured by echocardiography 42 days after TAC. Sham operation was used as a negative control. The graph shows a reduction in corrected LV Mass in Dox-treated (grey bar) vs untreated (black bar) mice.

FIG. 10 shows a TAC induced in rtTA-CnAbeta1 mice wherein doxycycline (Dox) was administered in the diet for 21 days, starting 21 days after TAC, and corrected LV mass was measured by echocardiography 42 days after TAC. The graph shows a reduction in left ventricular posterior wall thickness in Dox-treated (grey bar) vs untreated (black bar) mice.

FIG. 11 shows a TAC induced in rtTA-CnAbeta1 mice wherein doxycycline (Dox) was administered in the diet for 21 days, starting 21 days after TAC, and corrected LV mass was measured by echocardiography 42 days after TAC. The graph shows an improvement in ejection fraction in Dox-treated (grey bar) vs untreated (black bar) mice.

FIG. 12 shows a TAC induced in rtTA-CnAbeta1 mice wherein doxycycline (Dox) was administered in the diet for 21 days, starting 21 days after TAC, and wherein RNA was isolated from the left ventricle 42 days after TAC. Alpha skeletal actin (Acta1) expression was measured by qRT-PCR. The graph shows a reduction in Acta1 mRNA expression in Dox-treated (grey bar) vs untreated (black bar) mice.

FIG. 13 shows a TAC induced in rtTA-CnAbeta1 mice wherein doxycycline (Dox) was administered in the diet for 21 days, starting 21 days after TAC, and wherein RNA was isolated from the left. ventricle 42 days after TAC. Collagen I α1 (Col1a1) expression was measured by qRT-PCR. The graph shows a reduction in Col1a1 mRNA expression in Dox-treated (grey bar) vs untreated (black bar) mice.

### DETAILED DESCRIPTION OF THE INVENTION

### Definitions

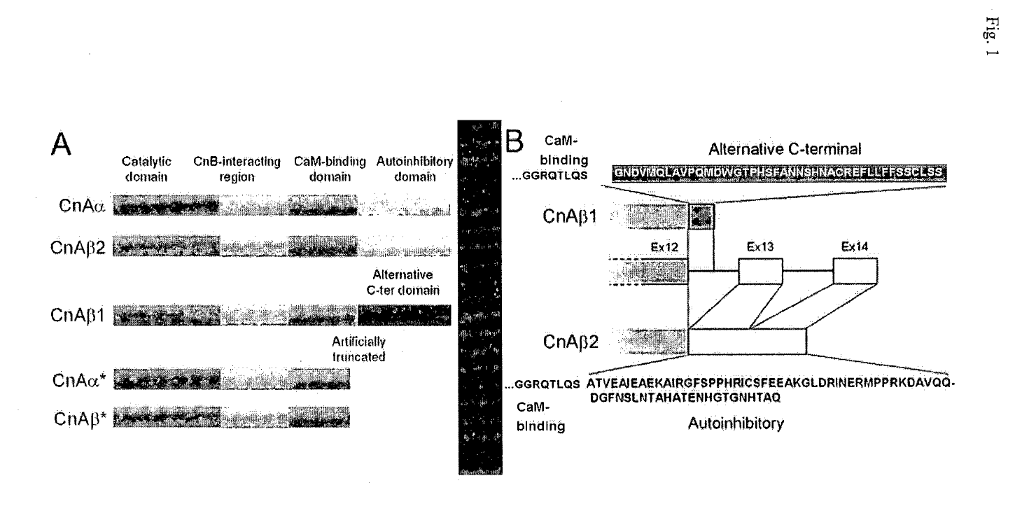

As used in the specification and the appended claims the term “calcineurin subunit Aβ1 isoform (CnAβ1)” must be understood as an isoform of CnAbeta (PPP3CB) which lacks the autoinhibitory domain present in all other naturally occuring Cn isoforms. This isoform was termed CnAbeta1, as opposed to the predominant CnAbeta2 isoform (generally known as CnAbeta). Instead of the typical CnA autoinhibitory domain, CnAbeta1 has a unique structure as a result of the translation of intron 12-13 (FIG. 1A). The human calcineurin subunit Ap β1 isoform (CnAβ1) is herein illustrated as having amino acid sequence SEQ ID No 1. The present definition of the calcineurin subunit Aβ1 isoform (CnAβ1) also includes interspecies and intraspecies variations. In this sense, a variant of the calcineurin subunit Aβ1 isoform (CnAβ1) is at least 70% homologous to SEQ ID No 1 based on amino acid identity, more preferably at least 80%, 85%, 90%, 95%, 97%, 98% or 99% homologous to SEQ ID No 1 based on amino acid identity.

As used in the specification and the appended claims the term “C-terminal domain of the calcineurin subunit Aβ1 isoform (CnAβ1)” must be understood as the C-terminal fraement o CnAβ1 represented here as arninoacid acid SEQ ID No 2. The present definition of the C-terminal domain of the calcineurin subunit Aβ1 isoform (CnAβ1) also includes interspecies and intraspecies variations. In this sense, a variant of the calcineurin subunit Aβ1 isoform (CnAβ1) is at least 70% homologous to SEQ ID No 2 based on amino acid identity, more preferably at least 80%, 85%, 90%, 95%, 97%, 98% or 99% homologous to SEQ ID No 2 based on amino acid identity.

As used in the specification and the appended claims the term “activator compound” must be understood as a compound capable of:
a. increasing the intracellular concentration of the calcineurin subunit Aβ1 isoform (CnAβ1) in the cardiomyocytes of a subject relative to that observed in the absence of the compound; and/or
b. increasing the intracellular concentration of a peptide comprising a fragment of the calcineurin subunit Aβ1 isoform (CnAβ1) in the cardiomyocytes of a subject relative to that observed in the absence of the compound, wherein said fragment consist of the C-terminal domain of the calcineurin subunit Aβ1 isoform (CnAβ1).

The activator compound can be a modulator of the intracellular expression of CnAβ1 that increases the amount of CnAβ1. In addition the activator compound can be a compound capable of increasing the intracellular concentration of CnAβ1, or of increasing the concentration of CnAβ1-encoding mRNA, and/or the posttranslational modification of CnAβ1 (if any).

The activator can either directly or indirectly affect the expression of CnAβ1. Activators can be identified, for example, through screening methods as described herein in the detailed description of the invention. An example of an activator compound which induces CnAβ1 expression by indirectly affecting the expression of CnAβ1 would be an inhibitor of an endogenous inhibitor of CnAβ1.

The term “increases” or “increasing” refers to increases above basal level. For example, basal levels are normal in vivo levels prior to, or in the absence of, addition of an activator compound.

The term “any part of the heart muscle” is defined as a single area of the heart, such as one ventricle for example, or any cell within the heart, preferably cardiomyocytes, or any intracellular location (site, compartment or microenvironment), or the entire heart.

In this specification and in the claims which follow, reference will be made to a number of terms which shall be defined to have the following meanings: “Optional” or “optionally” means that the subsequently described event or circumstance may or may not occur, and that the description includes instances where said event or circumstance occurs and instances where it does not.

As used herein, the terms “prevent”, “preventing” and “prevention” refer to the methods to avert or avoid a disease or disorder or delay the recurrence or onset of one or more symptoms of a disorder in a subject resulting from the administration of a prophylactic agent.

The term “pharmaceutically acceptable carrier” is intended to include formulation used to stabilize, solubilize and otherwise be mixed with active ingredients to be administered to living animals, including humans. This includes any and all solvents, dispersion media, coatings, antibacterial and antifungal agents, isotonic and absorption delaying agents, and the like, compatible with pharmaceutical administration. Except insofar as any conventional media or agent is incompatible with the active compound, such use in the compositions is contemplated.

The term “disease” as used herein is intended to be generally synonymous, and is used interchangeably with, the terms “disorder” and “condition” (as in medical condition), in that all reflect an abnormal condition of the body or of one of its parts that impairs normal functioning and is typically manifested by distinguishing signs and symptoms.

As used herein, the term “cardiac hypertrophy” refers to the process in which adult cardiac myocytes respond to stress through hypertrophic growth. Such growth is characterized by cell size increases without cell division, assembling of additional sarcomeres within the cell to maximize force generation, and an activation of a fetal cardiac gene program which in turn results in a thickening of the heart muscle (myocardium) which results in a decrease in size of the chamber of the heart, including the left and right ventricles.

As used herein the term “Pressure overload hypertrophy” or “Pathological cardiac hypertrophy induced by pressure overload” is a condition that occurs as a result of chronic increases in systolic wall stress. This may result from impedance to cardiac outflow (eg, subaortic stenosis, pulmonic stenosis) or increased vascular resistance (eg, systemic or pulmonary hypertension). The response of the myocardium to these conditions is concentric hypertrophy (ie, increased wall thickness of the affected chamber). It is noted that pressure overload—induced cardiac hypertrophy is different from those forms of cardiac hypertrophy produced as an adaptive response of the heart to ischemic heart failure with an acute thrombocytic coronary occlusion, i.e. myocardial infarction. In this sense, as already stated above, pressure overload hypertrophy induces concentric cardiac hypertrophy, as opposed to volume overload, which induces eccentric cardiac hypertrophy. Myocardial infarction induces an heterogeneous (uneven) combination of concentric and eccentric hypertrophy. Pressure overloadinduced cardiac hypertrophy is particularly understood in the present invention to be produced in aortic valve stenosis, chronic hypertension (hypertensive cardiomyopathy), pulmonary hypertension and hypertrophic cardiomyopathy.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the compound combination in treating the conditions or disorders described herein.

The phrase “therapeutically effective” is intended to qualify the amount of active ingredients used in the treatment of a disease or disorder. This amount will achieve the goal of reducing or eliminating the said disease or disorder.

The term “subject” means all mammals including humans. Examples of subjects includes, but are not limited to, humans, cows, dogs, cats, goats, sheep, pigs, and rabbits.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Description of the Invention

Heart disease and its manifestations, including coronary artery disease, myocardial infarction, congestive heart failure and cardiac hypertrophy, clearly presents a major health risk in the world today. The cost to diagnose, treat and support patients suffering from these diseases is well into the billions of dollars. Two particularly severe manifestations of heart disease are myocardial infarction and cardiac hypertrophy.

With respect to myocardial infarction, typically an acute thrombocytic coronary occlusion occurs in a coronary artery as a result of atherosclerosis and causes myocardial cell death. Because cardiomyocytes are terminally differentiated and generally incapable of cell division, they are generally replaced by scar tissue when they die during the course of an acute myocardial infarction. Scar tissue is not contractile, fails to contribute to cardiac function, and often plays a detrimental role in heart function by expanding during cardiac contraction, or by increasing the size and effective radius of the ventricle.

Cardiac hypertrophy is an adaptive response of the heart to virtually all forms of cardiac disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to heart failure and sudden death.

In the present invention, the inventors have surprisingly identified an isoform of the serine-threonine phosphatase calcineurin as a new target for the treatment of pathologic cardiac hypertrophy. Particularly cardiac hypertrophy induced as an adaptive response of the heart to mechanical load or hypertension, namely cardiac hypertrophy induced by pressure overload.

In this sense, it is well established that the serine-threonine phosphatase calcineurin is expressed in multiple tissues and consists of a catalytic A subunit and a regulatory B subunit.

Calcineurin B is encoded by two different genes and three different calcineurin A subunits (CnAα, CnAβ, and CnAγ) have been described in vertebrates. CnAα and CnAβ are widely expressed, whereas CnAγ expression is restricted to brain and testis. The physiological role of calcineurin was initially elucidated in T-cells in which elevations in cytoplasmic calcium concentrations promote the association of calmodulin and consequent activation of the enzyme. Calcineurin dephosphorylates transcription factors of the NFAT family, thereby unmasking nuclear localization signals, which in turn results in translocation of NFAT proteins to the nucleus and activation of immune response genes, such as interleukin-2.

This pathway has also been shown to be operative in cardiomyocytes. In this sense, it is well known that constitutive activation of calcineurin in transgenic mouse hearts is sufficient to induce massive cardiac enlargement and eventually heart failure. However, the inventors of the present invention have surprisingly found that the calcineurin subunit Aβ1 isoform (CnAβ1) does not only fail to induce such massive cardiac enlargement but, in contrast, it induces the reduction of such cardiac enlargement, in this sense it acts as an antihypertrophic agent in a vertebrate's heart muscle.

To demonstrate the above, the inventors of the present invention induced cardiac hypertrophy induced by pressure overload in mice through transaortic (TAC) constriction. TAC induces left ventricular pressure overload that results in cardiac hypertrophy. It is a good in vivo model of outflow tract obstruction (e.g, aortic stenosis) and systemic hypertension leading to increased cardiac after-load and cardiac hypertrophy.

TAC was performed in 2-6 month old wild type and transgenic mice, namely in αMHC-CnAbeta1 transgenic mice that overexpress the calcineurin variant CnAbeta1 (CnAβ1) specifically in post-mitotic cardiomyocytes.

Prior to induction of anesthesia, all mice were given buprenorphine (0.25 mg/kg subcutaneously). General anesthesia was induced by inhalation of sevofluorane (3-4% (v/v) in oxygen) and animals were restrained. Anesthesia was maintained by 1-1.5% isofluorane inhalation throughout the procedure. The chest hair was removed and the chest was disinfected with 70% ethanol followed by povidone iodine. Upper chest midline skin incision followed by median sternotomy was performed to open the chest cavity. The thymus was gently retracted until the transverse aorta was discernible. The loose suture (7-0 silk) was placed around the aorta and a 27 gauge blunted needle, between the right innominate artery and left common carotid artery. The suture was tightened to fully constrict the aorta and the needle was removed, thereby leading to partial constriction of the transverse aorta. The chest cavity, followed by the muscle and skin layer, was closed using 6-0 absorbable vicryl sutures. The chest was disinfected with povidone iodine and the animals were allowed to recover in a warm chamber. Analgesia (0.1 mg/kg buprenorphine) was given up to twice daily for 2-3 days following surgery. ‘Sham’ operation was similarly performed with the exception that the suture around the aorta was not constricted.

Both wild type and αMHC-CnAbeta1 transgenic mice showed a significant increase in heart weight to body weight ratio (HW/BW) 21 days after TAC compared to sham-operated mice (Example 2). Importantly, αMHC-CnAbeta1 mice showed significantly smaller hearts than wild type mice after TAC, as assessed by HW/BW (please refer to Example 2). To confirm these findings, the inventors carried out an echocardiographic analysis of wild type and transgenic mice that had undergone sham or TAC surgery. As shown in example 3, wild type mice presented increased left ventricular mass (LVMass corrected) 21 days after TAC, together with a thicker ventricular wall, measured both in the posterior wall (LVPW) and the interventricular septum (IVS). In contrast, αMHC-CnAbeta1 mice showed no significant increase in LVPW or IVS thickness and only a mild increase in LV mass. Importantly, all three hypertrophy parameters were decreased in αMHC-CnAbeta1 mice compared to wild type mice after TAC. To determine whether the attenuated hypertrophic response seen in αMHC-CnAbeta1 mice was paralleled by improved cardiac function, the authors carried out further echocardiographic analysis. Wild type mice suffered a decline in systolic function 21 days after TAC, as shown by fractional area change (FAC, example 4). However, αMHC-CnAbeta1 mice showed no functional decline and displayed significantly improved function compared to wild type mice after TAC. Together, these data demonstrate that CnAbeta1 overexpression in cardiomyocytes reduces pathological cardiac hypertrophy and improves cardiac function in a non-ischemic setting.

To further investigate the beneficial effects of CnAbeta1 overexpression on the hypertrophic heart, the inventors analyzed the mRNA expression of the heart failure markers atrial natriuretic factor (ANF) and alpha skeletal actin (Acta1) in the different mice and conditions. The inventors of the present invention observed a strong induction of ANF and Acta1 expression in wild type mice after TAC (example 5). Although increased expression of these markers was also found in αMHC-CnAbeta1 mice, they were significantly reduced compared to wild type mice, in agreement with their previous results.

Pathological cardiac hypertrophy is accompanied by the development of fibrosis. To investigate whether CnAbeta1 overexpression in cardiomyocytes has any effect of cardiac fibrosis, the inventors analyzed the expression of collagen I a1 (Col1a1) and the enzyme lysyl oxidase (Lox), responsible for ECM fibre crosslinking. As shown in example 6, Col1a1 and Lox tnRNA were strongly induced by TAC in wild type mice. Importantly, αMHC-CnAbeta1 mice showed reduced levels of both fibrotic markers after TAC compared to wild type mice.

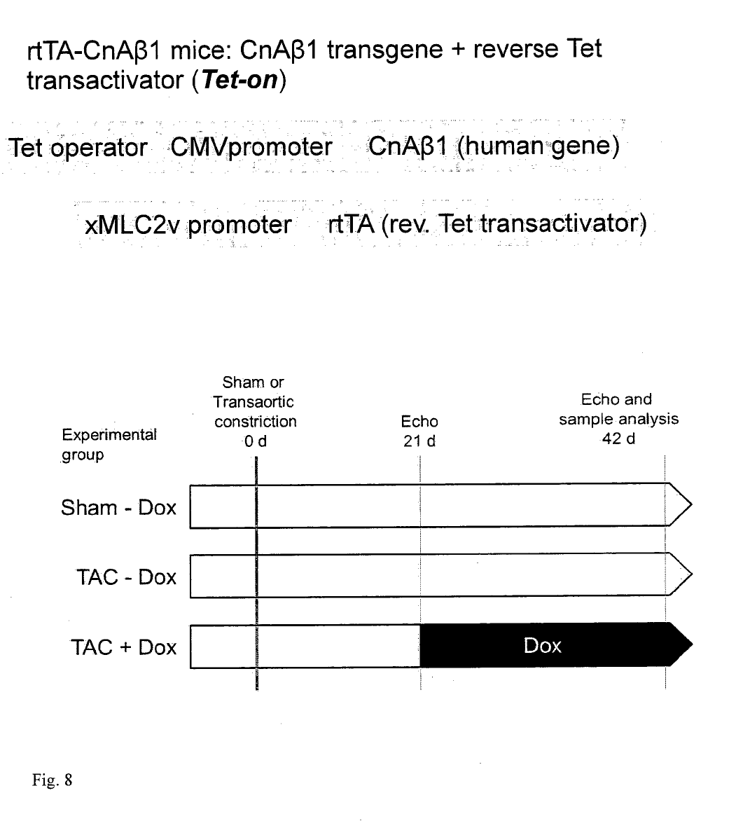

In order to further investigate the role of CnAbeta1 in the treatment of pathologic cardiac hypertrophy, the authors of the present invention induced the expression of CnAbeta1 in rtTA-CnAbeta1 mice by administering doxycycline in the diet (Dox) 21 days after transaortic banding. By this time, mice have developed pathological cardiac hypertrophy as a consequence of TAC, as evidenced by echocardiographic analysis. rtTA-CnAbeta1 are double transgenic mice. In one transgene, the expression of the reverse Tet transactivator (rtTA) is controlled by the myosin light chain 2v promoter from Xenopus tropicalis to ensure cardiomyocyte-specific expression. Administration of doxycicline in the diet activates the rtTA, which allows activation of the Tel operator in a second transgene and overexpression of CnAbeta1 from this second transgene (FIG. 8).

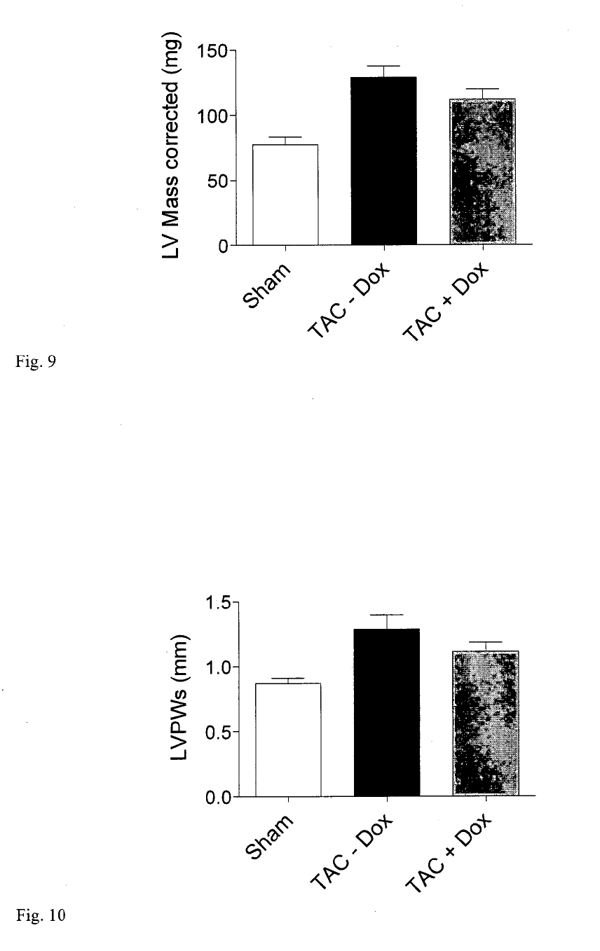

As illustrated in FIG. 8, rtTA-CnAbeta1 mice were treated with doxycycline in the diet starting 21 days after TAC (TAC+Dox). In contrast, the “TAC−Dox” group of rtTA-CnAbeta1 mice were not treated with doxycycline at any moment in time. Under these conditions, the authors of the present invention measured LV Mass (corrected) by echocardiography 42 days after surgery in rtTA-CnAbeta1 mice treated (TAC+Dox) and non treated (TAC−Dox) with doxycycline. Sham-operated rtTA-CnAbeta1 mice not treated with Dox were used as a negative control. As illustrated in FIG. 9, LV Mass was significantly lower in the TAC+Dox mouse group than in the TAC−DOX group, thus demonstrating that CnAbeta1 overexpression in cardiomyocytes after transaortic banding reduces cardiac hypertrophy.

In addition, left ventricle posterior wall thickness was measured by echocardiography 42 days after surgery (transaortic banding) in rtTA-CnAbeta1 mice treated and non treated with doxycycline in the diet. As illustrated in FIG. 10, the left ventricle posterior wall thickness is significantly lower in rtTA-CnAbeta1 mice treated with Dox (TAC+Dox) than in the untreated mice (TAC−DOX), thus demonstrating that CnAbeta1 overexpression in cardiomyocytes after transaortic banding reduces cardiac hypertrophy.

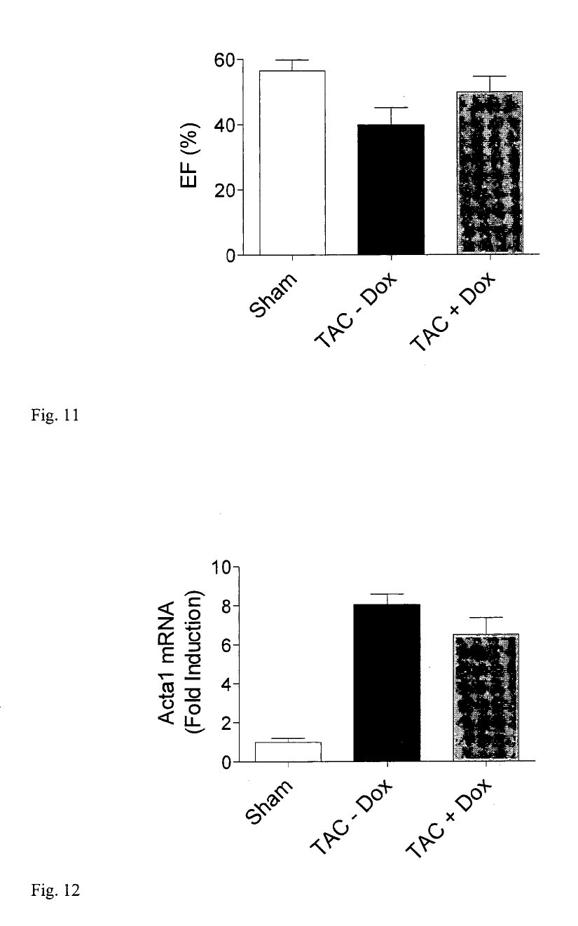

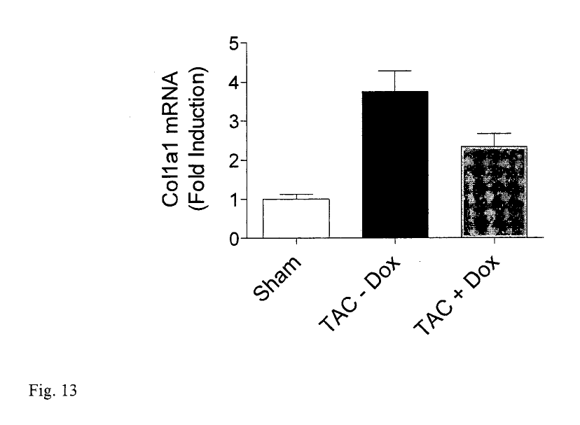

Lastly, ejection fraction, Acta1 (alpha skeletal actin) mRNA and Col1a1 (collagen I a1) mRNA were measured by echocardiography and qRT-PCR 42 days after surgery (transaortic banding) in rtTA-CnAbeta1 mice treated and non treated with doxycycline. As illustrated in FIGS. 11-13 all these parameters were improved in rtTA-CnAbeta1 mice treated with Dox (TAC+Dox) in comparison to rtTA-CnAbeta1 mice untreated with Dox (TAC−Dox), thus demonstrating that CnAbeta1 overexpression in cardiomyocytes after transaortic banding reduces cardiac hypertrophy.

In summary, these results demonstrate that overexpression of CnAbeta1 in post-mitotic cardiomyocytes reduces cardiac hypertrophy and improves cardiac function. Thus, CnAbeta1 has a therapeutic potential for the treatment of an undesirable cardiac hypertrophy effect, preferably for the treatment of a pathological cardiac hypertrophy induced by pressure overload such as the one produced in aortic valve stenosis and chronic hypertension.

Thus, these results demonstrate that increasing the intracellular CnAβ1 concentration reduces hypertrophic cardiomyopathy, particularly pressure overloadinduced cardiac hypertrophy. Consequently, increasing the intracellular CnAβ1 concentration is not useful for avoiding or averting (preventing) the occurrence of pathologic cardiac hypertrophy as an adaptive response of the heart to virtually all forms of cardiac disease but is surprisingly useful in treating said disease (pathologic cardiac hypertrophy) after its clinical manifestation.There was no way to infer, prior to the findings disclosed herein, that increased concentrations of CnAβ1 would have reduced cardiac hypertrophy. The results disclosed herein are thus the first to allow this interpretation.

Consequently, CnAβ1 has been identified as a crucial target to provide and develop new compositions suitable as drugs for treating an undesirable cardiac hypertrophy effect. In view of this, it is clear that there is a need in the art for agents that increase CnAβ1. These agents will provide an advance in the treatment of cardiac hypertrophy.

It is also noted that the increasing of particular intracellular fragments of CnAβ1 reduces hypertrophic cardiomyopathy, particularly pressure overloadinduced cardiac hypertrophy. These particular fragments of CnAβ1 include any fragment of the protein CnAβ1 comprising the C-terminal domain of CnAβ1. In this sense please note that the C-terminal domain of CnAβ1 is a unique peptide that shares no homology with any other known protein. It confers CnAβ1 functional properties that are not shared by other calcineurin isoforms, such as the interaction with the mTORC2 complex.

Therefore, disclosed herein are thus methods of increasing the expression or activity of the calcineurin subunit Aβ1 isoform (CnAβ1) or fragments thereof in any part of the heart muscle of a subject for treating an undesirable cardiac hypertrophy effect, the method comprising: a) identifying a subject who may benefit from CnAβ1 expression; and b) administering to the subject an activator of CnAβ1.

Consequently, a first aspect of the invention refers to a composition (from hereinafter composition of the invention) comprising a compound (from hereinafter activator compound of the invention) capable of
a. increasing the intracellular concentration of the calcineurin subunit Aβ1 isoform (CnAβ1) in the cardiomyocytes of a subject relative to that observed in the absence of the compound; and/or
b. increasing the intracellular concentration of a peptide comprising a fragment of the calcineurin subunit Aβ1 isoform (CnAβ1) in the cardiomyocytes of a subject relative to that observed in the absence of the compound, wherein said fragment consist essentially of the C-terminal domain of the calcineurin subunit Aβ1 isoform (CnAβ1);

for its use in the treatment of pathologic cardiac hypertrophy.

In a preferred aspect of the invention, said compound is capable of increasing the intracellular expression of the calcineurin subunit Aβ1 isoform (CnAβ1) in the cardiomyocytes of a subject relative to that observed in the absence of the compound.

In a more preferred aspect of the invention, the calcineurin subunit Aβ1 isoform (CnAβ1) is the human calcineurin subunit Aβ1 isoform (CnAβ1).

In another preferred embodiment of the first aspect of the invention, the pathologic cardiac hypertrophy is induced by a disease selected from the list consisting of hypertension, mechanical load, myocardial infarction, cardiac arrhythmias, endocrine disorders, and genetic mutations in cardiac contractile protein genes.

In another preferred aspect of the invention, the pathologic cardiac hypertrophy is not induced by an ischemic heart disease with acute thrombocytic coronary occlusion such as myocardial infarction. Preferably, the pathologic cardiac hypertrophy is induced by pressure overload. More preferably, the pathologic cardiac hypertrophy is induced by pressure overload caused by aortic valve stenosis, chronic hypertension (hypertensive cardiomyopathy), pulmonary hypertension or hypertrophic cardiomyopathy.

As already mentioned in the definitions above, activator compounds are thus those molecules that increase CnAβ1 functional activity or alter its intracellular distribution. In one embodiment, a compound is an activator compound when the compound reduces the incidence, severity or adverse consequences of cardiac hypertrophy relative to those observed in the absence of the compound. CnAβ1 concentration can be increased in any part of the heart muscle of a subject.

By “increases the intracellular expression” is meant increasing over the baseline, or compared to a control, by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39. 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or 2, 3, 4, 5, 6, 7, 8, 9, 10, 11, 12, 13, 14, 15, 20, 25, 30, 35, 40, 45, 50, or more fold.

Activator compounds can be identified by a method for screening a compound for the ability to activate CnAβ1, comprising contacting a cell with a compound suspected to activate CnAβ1; assaying the contents of the cells to determine the amount and/or biological activity of CnAβ1; and comparing the determined amount and/or biological activity of CnAβ1 to a predetermined level, wherein a change of said amount and/or biological activity of CnAβ1 is indicative for a compound that activates CnAβ1. Preferred is a method according to the invention, wherein the cell is a cardiomyocyte. Further preferred is a method according to the invention, wherein the amount of calcineurin Aβ1 mRNA is determined. In one preferred embodiment, screening is done by quantitative real-time RT-PCR using specific primers for each isoform.

In certain embodiments, such activator compound is selected from a peptide which comprises:
a) the sequence of SEQ ID No 1;
b) a fragment of (a), which at least comprises SEQ ID No 2; or
c) a variant of (a) or (b) which is at least 70% homologous to SEQ ID No 1 or to a fragment of SEQ ID No 1 which at least comprises SEQ ID No 2, more preferably at least 80%, 85%, 90%, 95%, 97%, 98% or 99% homologous to SEQ ID No 1 or to a fragment of SEQ ID No 1 which at least comprises SEQ ID No 2.

In a preferred aspect of the invention, the activator compotmd is a peptide which comprises SEQ ID No 1 or a peptide comprising a fragment of SEQ ID No 1 consisting of SEQ ID No 2. In another embodiment of the invention, the activator compound is a peptide which consists essentially of SEQ ID No 1 or of a peptide fragment of SEQ ID No 1 consisting essentially of SEQ ID No 2. In another embodiment, said peptide is SEQ ID No 2.

Over the entire length of sequence SEQ ID NO 1, a variant will preferably be at least 70% homologous to that sequence based on amino acid identity. Preferably, the peptide is at least 85 or 90% and more preferably at least 95, 97, 98 or 99% homologous to sequence SEQ ID NO 1 over the entire region, based on amino acid identity.

Fragments of the peptide sequence SEQ ID No 1 include SEQ ID No 2. Variants of this region will preferably be at least 70%, preferably at least 80% or 90% and more preferably 95% homologous to this region, based on amino acid identity.

Peptides of the invention may be modified for example by the addition of histidine residues to assist their identification or purification or by the addition of a signal sequence to promote their secretion from a cell where the polypeptide does not naturally contain such a sequence.

A peptide of the invention above may be labelled with a revealing label. The revealing label may be any suitable label which allows the peptide to be detected.

The peptides of the invention may be introduced into a cell, such a cardiomyocyte, by in situ expression of the peptide from a recombinant expression vector. The expression vector optionally carries an inducible promoter to control the expression of the polypeptide.

A peptide of the invention can be produced in large scale following purification by high pressure liquid chromatography (HPLC) or other techniques after recombinant expression.

In other embodiment of the invention, the activator compound is a nucleic acid encoding calcineurin CnAβ1 or the C-terminal domain thereof. The disclosed nucleic acids can be in the form of naked DNA or RNA.

In this sense, the activator compound can be a polynucleotide (from hereinafter polynucleotide of the invention) having a sequence selected from:
a) a DNA sequence encoding SEQ ID No 1 or the complementary sequence thereto;
b) a DNA sequence encoding a peptide comprising a fragment of SEQ ID No 1 consisting of SEQ ID No 2, or the complementary sequence thereto;
c) a sequence which selectively hybridises to a said sequence (a) or (b);
d) a DNA sequence encoding a peptide sequence which is at least 70% homologous to SEQ ID No 1, more preferably at least 80%, 85%, 90%, 95%, 97%, 98% or 99% homologous to SEQ ID No 1; or
e) a DNA sequence encoding a peptide sequence comprising an amino acid sequence which is at least 70% homologous to a fragment of SEQ ID No 1 consisting of SEQ ID No 2, more preferably at least 80%, 85%, 90%, 95%, 97%, 98% or 99% homologous to a fragment of SEQ ID No 1 consisting of SEQ ID No 2.

In a preferred aspect of the invention, the polynucleotide of the invention is a DNA sequence encoding SEQ ID No 1 or the complementary sequence thereto. In another preferred aspect of the invention, the polynucleotide of the invention is a DNA sequence encoding a peptide comprising a fragment of SEQ ID No 1 consisting of SEQ ID No 2, or the complementary sequence thereto.

In another aspect of the invention, the activator compound is a mRNA polynucleotide having a sequence selected from:
a) an mRNA sequence encoding SEQ ID No 1;
b) an mRNA sequence encoding a peptide fragment of SEQ ID No 1 consisting of

SEQ ID No 2;
c) an mRNA sequence encoding a peptide sequence which is at least 70% homologous to SEQ ID No 1, more preferably at least 80%, 85%, 90%, 95%, 97%, 98% or 99% homologous to SEQ ID No 1; or
d) an mRNA sequence encoding a peptide sequence which is at least 70% homologous to a fragment of SEQ ID No 1 consisting of SEQ ID No 2, more preferably at least 80%, 85%, 90%, 95%, 97%, 98% or 99% homologous to a fragment of SEQ ID No 1 consisting of SEQ ID No 2.

In a preferred aspect of the invention, the mRNA polynucleotide of the invention is an m A sequence encoding SEQ ID No 1 or an mRNA sequence encoding a peptide fragment of SEQ ID No 1 which at least comprises SEQ ID No 2.

The polynucleotides of the invention may include within them synthetic or modified nucleotides. A number of different types of modification to polynucleotides are known in the art. These include methylphosphate and phosphorothioate backbones, addition of acridine or polylysine chains at the 3′and/or 5′ends of the molecule. For the purposes of the present invention, it is to be understood that the polynucleotides described herein may be modified by any method available in the art.

Polynucleotides such as a DNA polynucleotide according to the invention may be produced recombinantly, synthetically or by any means available to those skilled in the art. They may also be cloned by standard techniques. The polynucleotides are typically provided in isolated and/or purified form.

In a further preferred aspect of the invention, the polynucleotides of the invention, such as those discussed above encoding calcineurin CnAβ1 or a fragment thereof comprising the C-terminal domain of CnAβ1, can be transported into the cardiomyocytes, without degradation, by plasmid or viral vectors that include a promoter yielding expression of the protein in the cells into which it is delivered.

Thus, in a further embodiment of the invention the activators compounds of the invention can comprise any of the disclosed above polynucleotides of the invention or a plasmid or vector capable of transporting or delivering said polynucleotides, preferably a viral vector.

Viral vectors arc, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry a nucleic acid coding for CnAβ1.

As a way of example only we shall illustrate herein below some viral vectors that can be use as delivery systems to practice the present invention, preferably for the delivery of nucleic acids encoding the calcineurin subunit Aβ1 isoform (CnAβ1) or a fragment thereof comprising the C-terminal domain of CnAβ1, into the cardiomyocytes of any part of the heart muscle.

Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of one to many genes depending on the size of each transcript, certainly for the delivery of a nucleic acid encoding the calcineurin subunit Aβ1 isoform (CnAβ1). It is preferable to include either positive or negative selectable markers along with other genes in the insert.

Since the replication machinery and packaging proteins in most retroviral vectors have been removed (gag, poi, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” Bio Techniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaurn, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

Particularly, adenoviruses can be use as a tool for gene therapy to treat cardiac related conditions as shown by Grines et al., 2003; Rissanen and Ylä-Herttuala, 2007, wherein administration of FGF4 to ischemic patients using adenovirus type 5 reduced ischemic defect size and increased exercise capacity without any significant safety risk. Similarly, delivery of VEGF to patients with ischemic refractory heart disease was safe and it improved exercise capacity, compared to placebo, although it did not change myocardial perfusion (Rissanen and Ylä-Herttuala, 2007; Stewart et al., 2006). These trials show that gene therapy with adenoviruses is safe, although its efficacy needs to be improved

Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B 19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression.

The activators compounds can comprise, in addition to the disclosed polynucleotides of the invention, plasmid or vectors or the peptides of the invention, for example, lipids such as liposomes, such as cationic liposomes (e.g., DOTMA, DOPE, DC-cholesterol) or anionic liposomes.

Liposomes can further comprise proteins to facilitate targeting a particular cell, if desired. Administration of a composition comprising a compound and a cationic liposome can be administered to the blood afferent to a target organ. Furthermore, the activator can be administered as a component of a microcapsule that can be targeted to specific cell types, such as cardiomyocytes, or where the diffusion of the compound or delivery of the compound from the microcapsule is designed for a specific rate or dosage.

In addition the activator compounds of the invention can further comprise an antisense DNA sequence that hibridises a regulatory element in the PPP3CB gene (calcineurin A beta) and promotes an increase of CnAβ1 expression.

The DNA polynucleotides of the invention, such as the ones disclosed above, that are delivered to cardiomyocytes can be integrated into the host cell genome, typically through integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can become integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

The activator compounds herein disclosed can be administered in a pharmaceutically acceptable carrier and can be delivered to the subject's cardiomyocytes in vivo and/or ex vivo by a variety of mechanisms well known in the art as commented above.

If ex vivo methods are employed, cells or tissues can be removed and maintained outside the body according to standard protocols well known in the art. The activator compounds can be introduced into the cells, preferably cardiomyocytes, via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

The activators compounds of the present invention can be used in conjunction with another treatment method. Examples of other treatments that can be used in conjunction with those disclosed herein include, but are not limited to, catecholamines and catecholamine receptor agonists and antagonists (including [alpha]-and [beta]-adrenergic receptor agonists and antagonists), vasopressin receptor agonists and antagonists, organic nitrates, activators of soluble and particulate guanylyl cyclases, natriuretic peptides or other components that bind to natriuretic peptide receptors and stimulate or inhibit guanylyl cyclase activity, renin inhibitors, angiotensin-converting enzymes inhibitors, angiotensin receptor antagonists.

Additionally, provided herein is a method of increasing or enhancing the chances of survival of a subject with cardiac hypertrophy, preferably with pressure overload induced cardiac hypertrophy, comprising administering to a subject in need thereof an effective amount of an activator compound, thereby increasing or enhancing the chances of survival of the subject treated by a certain period of time, for example, by at least 10 days, 1 month, 3 months, 6 months, 1 year, 1.5 years, 2 years, 3 years, 4 years, 5 years, 8 years, or 10 years. The increase in survival of a subject can be defined, for example, as the increase in survival of a preclinical animal model by a certain period of time, for example, by at least 10 days, 1 month, 3 months, 6 months, or 1 year, or at least 2 times, 3 times, 4 times, 5 times, 8 times, or 10 times, more than a control animal model (that has the same type of disease) without the treatment with the inventive method. Optionally, the increase in survival of a mammal can also be defined, for example, as the increase in survival of a subject with heart disease by a certain period of time, for example, by at least 10 days, 1 month, 3 months, 6 months, 1 year, 1.5 years, 2 years, 3 years, 4 years, 5 years, 8 years, or 10 years more than a subject with the same type of heart disease but without the treatment with the inventive method. The control subject may be on a placebo or treated with supportive standard care such as chemical therapy, biologics and/or radiation that do not include the inventive method as a part of the therapy.

Also provided herein is a method of increasing or enhancing the clinical status and perception of well-being of a subject with cardiac hypertrophy, preferably with pressure overload induced cardiac hypertrophy, comprising administering to a subject in need thereof an effective amount of an activator compound, thereby increasing or enhancing the chances of survival of the subject treated by a certain period of time,

The present treatment methods also include a method to increase the efficacy of other agents given for the same disease, comprising administering to a subject in need thereof an effective amount of an activator compound; and, optionally, a pharmaceutically acceptable carrier, thereby increasing the efficacy of the other agent or agents.

In any case, the compositions comprising the activator compound can be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Effective dosages and schedules for administering the compositions comprising the activator compound disclosed herein may be determined empirically, and making such determinations is within the skill in the art. The dosage ranges for the administration of the compositions are those large enough to produce the desired anti-hypertrophic effect in the disorder. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

The following examples merely serve to illustrate the present invention.

### EXAMPLES

### Example 1

### Materials and Methods

### Example 1.1

### Mice

The mice used herein were αMHC-CnAbeta1 transgenic mice that overexpress the calcineurin variant CnAbeta1 (CnAβ1) specifically in cardiomyocytes under the control of the alpha myosin heavy chain promoter. The production of these mice is illustrated in Felkin L E, Narita T, Germack R, Shintani Y, Takahashi K. Sarathchandra P. López-Olañeta M M, Gómez-Salinero J M et al (2011) Calcineurin splicing variant CnAβ1 improves cardiac function after myocardial infarction without inducing hypertrophy. Circulation 123:2838-2847.

### Example 1.2

### Cardiac Hypertrophy

Cardiac hypertrophy was induced in mice through transaortic (TAC) constriction as described in Shimano M et al, Cardiac Myocyte-specific Ablation of Follistatin-like 3 Attenuates Stress-induced Myocardial Hypertrophy. J Biol Chem 286:9840-9848.

### Example 1.3

### EchocarDiographic Analysis

Cardiac function was measured by trans-thoracic echocardiography before and 3 weeks after TAC or Sham procedure. A Vevo 2200 machine (Visualsonics) was used on mice under general anesthesia with sevoflurane. M-mode and two dimensional images were obtained at the papillary muscle levels of the cardiac chamber. The left ventricular (LV) chamber dimensions during systole and diastole were marked and the different parameters were calculated. Three independent measurements were taken per mouse and average values were used.

### Example 1.4

### Tissue Sampling

Three weeks after TAC or Sham surgery, mice were weighed (body weight—BW) and general anesthesia was induced as described above. The chest cavity was opened and the heart was removed by dissection. The heart weight (HW) was measured and the HW/BW ratio was calculated. The heart was cut transversely at the papillary muscle level. The upper part, including the atria was placed in a microtube containing 10% formalin for fixation of the tissue. The LV was dissected out, divided into two pieces and snap-frozen in liquid nitrogen.

### Example 1.5

### RNA Isolation

Myocardial samples from mouse ventricles were snap frozen in liquid nitrogen and stored at-80° C. RNA was isolated by using the RNeasy Mini kit (Qiagen, UK-#74106) according to the manufacturer's instructions. Frozen samples were placed directly into RLT buffer and homogenized using an Ultra Turrax homogenizer (IKA). Homogenized lysates (500 μl) were diluted by adding 983 μl double-distilled water (ddH₂O) and 17 μl proteinase K (20 μg/μl concentration) was added to the lysate. They were incubated at 55° C. for 10 minutes in a water bath. The tissue debris was pelleted by centrifugation at 13,000 rpm for 3 minutes at room temperature. The supernatant was transferred to a fresh 2 ml microtube and 0.5 volumes of 100% ethanol were added and mixed by pipetting. To adsorb RNA from the sample onto the membrane, 700 μl of sample was added to an RNeasy mini column placed in a 2 ml collection tube and centrifuged for 15 seconds at 13,000 rpm. The flow-through was discarded and the step repeated until the entire sample was passed through the column for adsorption. Wash buffer (RW1) was added to the column (700 μl) and incubated for 5 minutes at room temperature followed by centrifugation for 15 seconds at 13,000 rpm. The flow-through was discarded and 80 μl was DNase I mixture (concentration=0.34 U/μl; Qiagen, UK-#79254) was carefully added directly on top of the membrane. The column was left at room temperature for 15 minutes, followed by another wash with 700 μl of wash buffer RW 1. The column was transferred to a new collection tube and washed with 500 μl ethanol-containing wash buffer RPE by centrifugation at 13,000 rpm for 15 seconds followed by another wash with RPE by centrifugation for 2 minutes. To ensure no carry-over of ethanol to the eluted sample, the collection tube was changed and the membrane dried by centrifugation for 1 minute at 13,000 rpm. The flow-through and collection tube were discarded. To elute the bound RNA sample from the column, the column was transferred to a fresh 1.5 ml microtube and 30 μl of RNase-free water was pipetted on the membrane avoiding direct contact. These were left at room temperature for 10 minutes before centrifugation for 1 minute at 13,000 rpm. Another elution was carried out similarly by adding 20 μl RNase-free water to the column and centrifugation. The column was discarded and the RNA sample was quantified and stored at-80° C. until required. RNA obtained from cells or tissues was quantified by spectrophotometry by measuring the absorbance at 260 nm (A_{260 nm}). The RNA concentration was calculated as: RNA (μg/ml)=A_{260 nm} X dilution factor X 40. For estimation of sample purity, the RNA samples were diluted 1:12 in TE buffer pH 8.0. A_{260 nm} and A_{280 nm} were measured and the ratio of A_{260/280} was calculated. Pure RNA samples usually have an A_{260/280} ratio of 2.0-2.1.

### Example 1.6

### qRT-PCR Analysis

For cDNA synthesis, 0.2 μg RNA were used. RNA samples were always thawed and kept on ice. The High Capacity cDNA Reverse Transcription Kit (Applied Biosystems) was used and a master mix of all the reagents was prepared on ice. To every tube, 13.05 μl of the master mix was added, followed by a total of 200 ng RNA sample and RNase-free water to make up the total volume to 20 μl as detailed in Table 1:

TABLE 1
cDNA synthesis reaction setup

| | | | Amount (μl) for |
|---|---|---|---|
| Reagent | Final concentration | | 20 μl reaction |
| RT Buffer, 10X | 1X | | 2 |
| MgCl₂ 25 mM | 5.5 | mM | 4.4 |
| dNTP mix (10 mM) | 500 μM each dNTP | | 4 |
| Random hexamers 50 μM | 2.5 | μM | 1 |
| RNase inhibitor | 0.4 | U/μl | 0.4 |
| MultiScribe ™ Reverse | 1.25 | U/μl | 1.25 |
| Transcriptase | | | |
| RNase-free water | — | | |
| RNA sample | 200 | ng | 6.95 |

The cycling conditions used for eDNA synthesis are as follows:

TABLE 2
cDNA synthesis reaction conditions

| Step | Temperature (° C.) | Time (minutes) |
|---|---|---|
| Primer binding | 25 | 10 |
| Extension | 48 | 30 |
| Enzyme denaturation | 95 | 5 |

cDNA samples were stored at −20° C. until required.

Quantitative real-time PCR (qRT-PCR) was performed using FAM-labelled off-the-shelf TaqMan Gene Expression assays (Applied Biosystems, UK). Amplification of an endogenous reference gene (18S rRNA or GA PDH mRNA) was carried out at the same time using primers and probe labeled with VIC reporter dye and TAMRA quencher dye. This allows measurement of Ct (Cycle Threshold) values for target gene and control in the same tube and the ΔCt is calculated as the difference between Ct of target gene and Ct of endogenous reference genes. From this, the relative expression of the gene is calculated using the comparative Ct method: Relative gene expression=̂ (−Δ[ΔCt]); where Δ [ΔCt] is the difference between ΔCt of test sample and ΔCt of blank/calibrator sample. Thus the abundance of the target gene is calculated, normalized to an endogenous reference gene like 18S.

The qRT-PCR reaction was set-up as a 10 μl reaction using 1 μl cDNA prepared from 200 ng RNA. TaqMan Gene Expression 2× master mix and target gene assays as well as 18S rRNA assay were purchased from Applied Biosystems. Real-time PCR was performed using the AB 7900-FAST-384 cycler (Applied Biosystems). The cycling conditions are shown in Table 3. Data were collected and analyzed after setting the baseline and threshold for each reporter dye manually. Relative gene expression was calculated by the comparative Ct method.

TABLE 3
Real-time PCR cycling conditions

| | Temperature | Time | |
|---|---|---|---|
| Step | (° C.) | (minutes) | Purpose |
| 1^{st} Hold | 50 | 2 | UDG incubation |
| 2^{nd} Hold | 95 | 10 | Activates DNA polymerase |
| PCR: 40 cycles | 95 | 15 | Denaturation |
| | 60 | 1 | Annealing and extension* |

*Primers anneal during the 60° C. incubation while probes anneal as the temperature reduces from 95° C. to 60° C.

### Example 2.

### Overexpression of CnAbeta1 in Cardiomyocytes Attenuates Heart Enlargement Following Transaortic Constriction

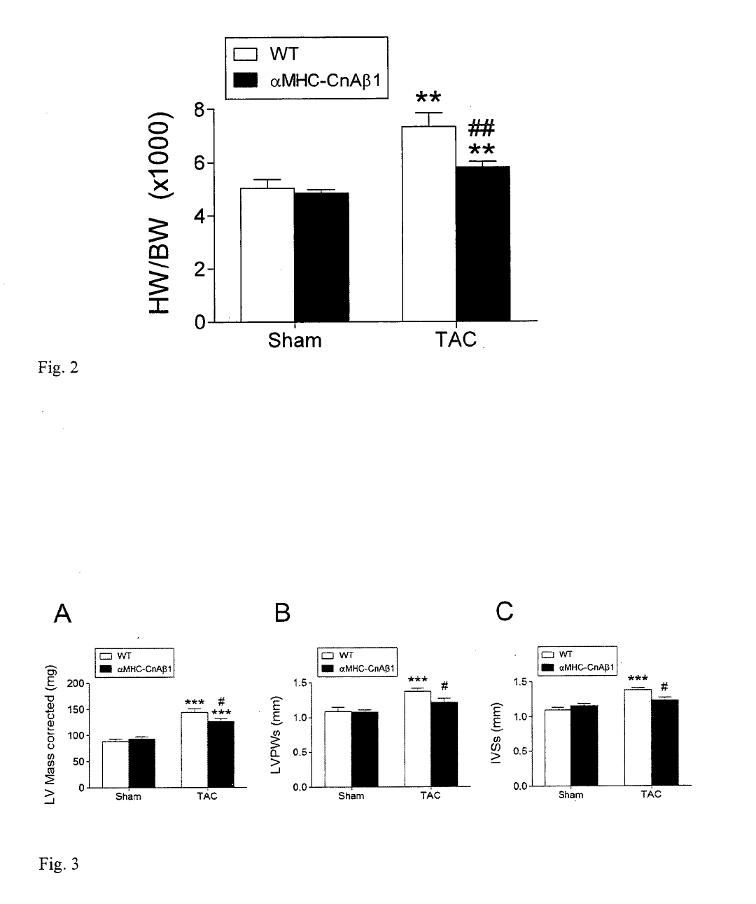

TAC was induced in wild type (WT) and αMHC-CnAbeta1 mice and animals were sacrificed 21 days later. The heart weight and body weight were measured and expressed as average of 1000× the HW/BW ratio±SD. Statistical significance was determined with a 2-way Anova test with Bonferroni post-test. **p<0.005, TAC vs. Sham for each mouse type; ##p<0.01, αMHC-CnAbeta1 TAC vs. WT TAC. n=10-18 animals per group. These results are illustrated in FIG. 2

### Example 3.

### CnAbeta1 Reduces Cardiac Hypertrophy

TAC was induced in wild type (WT) and αMHC-CnAbeta1 mice and corrected LV mass (A), systolic left ventricle posterior wall thickness (B, LVPWs) and systolic interventricular septum thickness (C, IVSs) were measured by echocardiography 21 days later. Data are expressed as average±SD. Statistical significance was determined with a 2-way Anova test with Bonferroni post-test. ***p<0.0005, TAC vs. Sham for each mouse type; #p<0.05, αMHC-CnAbeta1 TAC vs. WT TAC. n=13-18 animals per group. These results are illustrated in FIG. 3.

### Example 4.

### Overexpression of CnAbeta1 in the Hypertrophic Heart Improves Cardiac Function

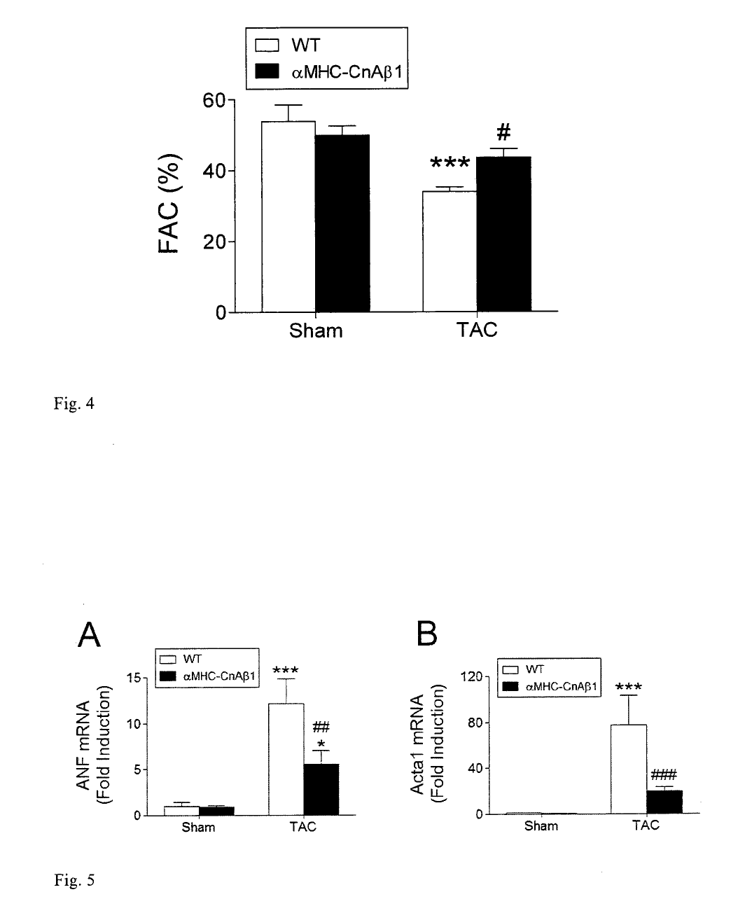

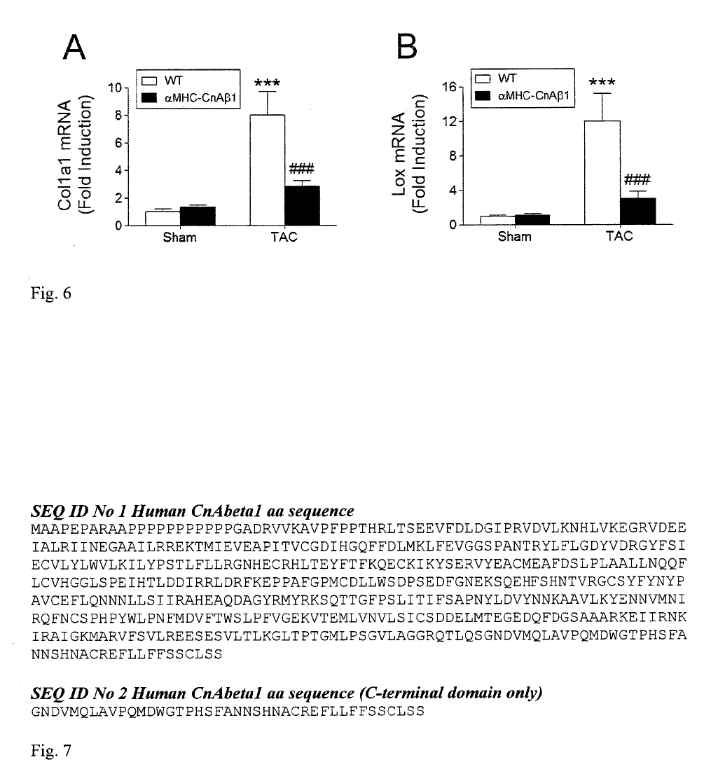

TAC was induced in wild type (WT) and αMHC-CnAbeta1 mice and fractional area change (FAC) was measured by echocardiography 21 days later. Data are expressed as average±SD. Statistical significance was determined with a 2-way Anova test with Bonferroni post-test. ***p<0.0005, TAC vs. Sham for each mouse type; #p<0.05, αMHC-CnAbeta1 TAC vs. WT TAC. n=13-18 animals per group. These results are illustrated in FIG. 4.

### Example 5.

### Expression of Heart Failure Markers After TAC is Significantly Lower in αMHC-CnAbeta1 Mice Than in Wild Type Mice

TAC was induced in wild type (WT) and αMHC-CnAbeta1 mice and RNA was isolated from the left ventricle 21 days later. Atrial natriuretic peptide (A, ANF) and alpha skeletal actin (B, Acta1) mRNA were measured by qRT-PCR. Data are expressed as average±SD. Statistical significance was determined with a 2-way Anova test with Bonferroni post-test. *p<0.05, ***p<0.0005, TAC vs. Sham for each mouse type; ##p<0.005, ###p<0.0005, αMHC-CnAbeta1 TAC vs. WT TAC. n=6-17 animals per group. These results are illustrated in FIG. 5.

### Example 6.

### Overexpression of CnAbeta1 in Cardiomyocytes Reduces Fibrosis in the Hypertrophic Heart

TAC was induced in wild type (WT) and αMHC-CnAbeta1 mice and RNA was isolated 21 days later from the left ventricle. Collagen I α1 (A, Col1a1) and lysyl oxidase (B, Lox) mRNA were measured by qRT-PGR. Data are expressed as average±SD. Statistical significance was determined with a 2-way Anova test with Bonferroni post-test. ***p<0.0005, TAC vs. Sham for each mouse type; ###p<0.0005, αMHC-CnAbeta1 TAC vs. WT TAC. n=7-16 animals per group.

1. A composition comprising a compound capable of
a. increasing the intracellular concentration of the calcineurin subunit Aβ1 isoform (CnAβ1) in the cardiomyocytes of a subject relative to that observed in the absence of the compound; and/or
b. increasing the intracellular concentration of a peptide comprising a fragment of the calcineurin subunit Aβ1 isoform (CnAβ1) in the cardiomyocytes of a subject relative to that observed in the absence of the compound, wherein said fragment consist of the C-terminal domain of the calcineurin subunit Aβ1 isoform (CnAβ1);
for its use in the treatment of pathologic cardiac hypertrophy.

2. The composition for use according to claim 1, wherein said compound is capable of increasing the intracellular expression of the calcineurin subunit Aβ1 isoform (CnAβ1) in the cardiomyocytes of a subject relative to that observed in the absence of the compound.

3. The composition for use according to any one of claim 1 or 2, wherein the calcineurin subunit Aβ1 isoform (CnAβ1) is the human calcineurin subunit Aβ1 isoform (CnAβ1).

4. The composition for use according to any one of the precedent claims, wherein the pathologic cardiac hypertrophy is not induced by an ischemic heart disease with acute thrombocytic coronary occlusion such as myocardial infarction.

5. The composition for use according to any one of claims 1 to 4, wherein the pathologic cardiac hypertrophy is induced by pressure overload.

6. The composition for use according to claim 5, wherein the pathologic cardiac hypertrophy is induced by pressure overload caused by aortic valve stenosis, chronic hypertension (hypertensive cardiomyopathy), pulmonary hypertension or hypertrophic cardiomyopathy.

7. The composition for use according to claim 6, wherein the cardiac hypertrophy is induced by pressure overload caused by aortic valve stenosis.

8. The composition for use according to any of claims 1-3, wherein the cardiac hypertrophy is induced by a disease selected from the list consisting of hypertension, mechanical load, myocardial infarction, cardiac arrhythmias, endocrine disorders, and genetic mutations in cardiac contractile protein genes.

9. The composition for use according to any of claims 1 or 3 to 8, wherein the compound is a peptide which comprises:
a. the sequence of SEQ ID No 1; or
b. a fragment of (a) consisting of SEQ ID No 2.

10. The composition for use according to any of claims 1 to 8, wherein the compound is a polynucleotide having a sequence selected from:
a. a sequence encoding SEQ ID No 1 or the complementary sequence thereto;
b. a sequence encoding a peptide comprising a fragment of SEQ ID No 1, wherein said fragment consists of SEQ ID No 2, or the complementary sequence thereto; or
c. a sequence which selectively hybridises to a said sequence (a) or (b).

11. The composition for use according to claim 10, wherein the polynucleotide sequence is selected from the list consisting of:
a. a sequence encoding SEQ ID No 1 or the complementary sequence thereto; or
b. a DNA sequence encoding a peptide fragment of SEQ ID No 1 consisting of SEQ ID No 2, or the complementary sequence thereto.

12. The composition for use according to any of claims 1 to 8, wherein the compound is a vector or plasmid capable of transporting or delivering a polynucleotide sequence as defined in any of claim 10 or 11 into the cardiomyocytes of a subject.

13. The composition for use according to claim 12, wherein the vector is a viral vector encoding for a polynucleotide sequence as defined in any of claim 10 or 11, optionally selected from the list consisting of adenoviral, retroviral or Adeno-Associated Viral Vectors.

14. The composition for use according to any of claims 1 to 8, wherein the composition comprises cells that recombinantly express CnAβ1.

15. The composition for use according to any of the precedent claims, wherein the composition is a pharmaceutical composition optionally comprising a pharmaceutically acceptable carrier.

16. The pharmaceutical composition for use according to claim 15, wherein this use is in combination therapy with a further active pharmaceutical ingredient.

17. Method for screening a compound for the ability to activate CnAβ1, comprising:
a. contacting a cell with a compound suspected to activate CnAβ1;
b. assaying the contents of the cells to determine the amount and/or biological activity of CnAβ1; and
c. comparing the determined amount and/or biological activity of CnAβ1 to a predetermined level, wherein a change of said amount and/or biological activity of CnAβ1 is indicative for a compound that activates CnAβ1.

18. The method of claim 17 wherein the cell is a cardiomyocyte.

19. A method for producing a compound capable of activating CnAβ1, which comprises:
a. Screening for a compound for the ability to activate CnAβ1 according to any of claim 17 or 18; and
b. Providing the compound identified in step (a).


SEQUENCE LISTING (4 sequences)

SEQ ID NO: 1 - protein, 496 aa, Homo sapiens
  PEPTIDE (1)..(496): Human CnAbeta1 aa sequence
MAAPEPARAAPPPPPPPPPPPGADRVVKAVPFPPTHRLTSEEVFDLDGIPRVDVLKNHLVKEGRVDEEIALRIINEGAAILRREKTMIEVEAPITVCGDIHGQFFDLMKLFEVGGSPANTRYLFLGDYVDRGYFSIECVLYLWVLKILYPSTLFLLRGNHECRHLTEYFTFKQECKIKYSERVYEACMEAFDSLPLAALLNQQFLCVHGGLSPEIHTLDDIRRLDRFKEPPAFGPMCDLLWSDPSEDFGNEKSQEHFSHNTVRGCSYFYNYPAVCEFLQNNNLLSIIRAHEAQDAGYRMYRKSQTTGFPSLITIFSAPNYLDVYNNKAAVLKYENNVMNIRQFNCSPHPYWLPNFMDVFTWSLPFVGEKVTEMLVNVLSICSDDELMTEGEDQFDGSAAARKEIIRNKIRAIGKMARVFSVLREESESVLTLKGLTPTGMLPSGVLAGGRQTLQSGNDVMQLAVPQMDWGTPHSFANNSHNACREFLLFFSSCLSS

SEQ ID NO: 2 - protein, 41 aa, Homo sapiens
  PEPTIDE (1)..(41): C-terminal domain only of the human CnAbeta1 aa sequence.
GNDVMQLAVPQMDWGTPHSFANNSHNACREFLLFFSSCLSS

SEQ ID NO: 3 - protein, 49 aa, Homo sapiens
  PEPTIDE (1)..(49): Artificial peptide sequence
GGRQTLQSGNDVMQLAVPQMDWGTPHSFANNSHNACREFLLFFSSCLSS

SEQ ID NO: 4 - protein, 77 aa, Homo sapiens
  PEPTIDE (1)..(77): Artificial sequence
GGRQTLQSATVEAIEAEKAIRGFSPPHRICSFEEAKGLDRINERMPPRKDAVQQDGFNSLNTAHATENHGTGNHTAQ